(19) United States
(12) Patent Application Publication
(10) Pub. No.: US 2003/0082793 A1
(43) Pub. Date: May 1, 2003

(54) Method for preparation of physiologically active water-base extract and method for potentiating the activity thereof

(76) Inventors: Takaaki Maekawa, Inashiki-gun (JP); Hiroko Isoda, Tsukuba-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/228,112

(22) Filed: Aug. 27, 2002

(30) Foreign Application Priority Data
Aug. 30, 2001 (JP) 2001-262244

(51) Int. Cl. C12N 1/14

(52) U.S. Cl. 435/254.1

(57) ABSTRACT

A physiologically active extract is prepared by disintegrating mycelial bodies of agaricus fungus belonging to the basidiomycetous order of matsutake to give a disintegrated material, heating a slurry containing the disintegrated material or keeping a slurry containing the disintegrated material with addition of ethyl alcohol or a mixture of ethyl alcohol and water and removing solid materials from the slurry. The physiologically active extract is useful for therapy of dementia and Alzheimer's disease.

### BACKGROUND OF THE INVENTION

The present invention relates to a method for the preparation of a novel physiologically active extract having neurocyte differentiation-inductive activity and acetylcholinesterase activity and having usefulness for therapy of dementia, Alzheimer's disease and others.

Agaricus (Agaricus Blazei Murill), having origin in Brazil and belonging to the basidiomycetous order of matsutake, is used for food as a health food and, besides, is widely employed as a medicinal material.

Since a great variety of ingredients are contained in agaricus, however, explication of the composition thereof has not yet been done sufficiently. The only report heretofore is that, among the water-soluble ingredients contained in the agaricus, β-D-glucan exhibits an anticancer activity.

On the other hand, it is known that a physiologically active substance having a neurocyte differentiation-inductive activity and an acetylcholinesterase activity is useful for therapy of dementia, Alzheimer's disease and others. Nevertheless, existence of a physiologically active substance having a neurocyte differentiation-inductive activity and acetylcholinesterase activity in the ingredients of basidiomycetous agaricus was absolutely unknown up to now.

### SUMMARY OF THE INVENTION

The present invention has been completed, under such situations, with an object to provide a method for the preparation of a novel physiologically active extract having usefulness for the therapy of dementia and Alzheimer's disease from basidiomycetous agaricus as the starting material.

The inventors have continued extensive investigations on the ingredients in the agaricus fungus belonging to the basidiomycetous order of matsutake and have arrived at a discovery that a physiologically active substance having a neurocyte differentiation-inductive activity and acetylcholinesterase activity exists therein and that the effectiveness of this physiologically active substance is potentiated when used in combination with soybean choline leading to completion of the present invention on the base of these discoveries.

Thus, the present invention provides a method for the preparation of a physiologically active extract which comprises disintegrating mycelial bodies of agaricus fungus belonging to the basidiomycetous order of matsutake to give a disintegrated material, heating a slurry containing the disintegrated material and removing solid matters from the slurry as well as a method for the preparation of a physiologically active extract which comprises disintegrating mycelial bodies of agaricus fungus to give a disintegrated material, holding a slurry containing the disintegrated material with admixture of ethyl alcohol or a mixture of ethyl alcohol and water to effect an extraction treatment at a temperature lower than the boiling point thereof and removing solid matters from the slurry.

### EMBODIMENTS OF THE INVENTION

The agaricus fungus used in the present invention is not particularly limitative relative to the derivation thereof and any of wild-grown ones, artificially grown ones, those by culturing mycelial bodies and others can be used. There are also no particular limitations to the agaricus relative to the gathering season, years of growing, method of culturing, length of culturing term and so on.

While a difference in the composition of the culture medium of the aforementioned agaricus fungus may result in a difference of the activity, any of them can be used in the present invention. They can be used singly or can be used as a combination of a plurality thereof. Exhibition of a higher activity can be expected when an agaricus of high activity is used in combination.

According to the method of the present invention, a physiologically active extract having a neurocyte differentiation-inductive activity and acetylcholinesterase activity can be obtained by subjecting mycelial bodies of agaricus fungus to a hot-water extraction treatment or extraction treatment with ethyl alcohol or water-ethyl alcohol.

When the mycelial bodies of agaricus fungus used there are those by culturing, they can possibly be those by tank culturing for 3 to 14 days in a culture medium with addition of a single kind or two kinds in combination of disintegrated materials of, for example, wheat bran, bagasse, pinewood chips, pinewood fruits, wheat, foxtail millet, rice, rice straws, wheat straws and the like in a range of 0.2 to 3 g/liter.

In conducting the hot-water extraction treatment, it is preferable when the mycelial bodies of agaricus are by the tank culturing that they are disintegrated as such by means of an ultrasonic treatment and, in the case of mycelial bodies by solid culturing, the mycelial bodies of agaricus as gathered are thoroughly washed to remove dirty matters adhering to the surface and living bodies followed by cutting and disintegrating, preferably, by means of an ultrasonic treatment. According to need, a treatment with a homogenizer is further undertaken. In this case, it is optional, if so desired, that the disintegration treatment is preceded by air drying or a freeze-drying treatment.

Nextly, a slurry consisting of the disintegrated material of the agaricus mycelial bodies thus obtained and water is boiled under agitation to effect an extraction treatment. The amount of water used here is, though not particularly limitative, selected usually in the range of 5 to 20 times by mass relative to the amount of the disintegrated material of the agaricus mycelial bodies. After the extraction treatment, solid matters are removed by centrifugal separation to give a hot-water extract. It is preferable to repeat this extraction procedure several times.

When the extraction treatment is carried out with ethyl alcohol or water-ethyl alcohol, on the other hand, a disintegrated material of the agaricus mycelial bodies is first prepared in the same manner as in the above-described hot-water extraction treatment. Nextly, the slurry containing this disintegrated material of the agaricus mycelial bodies is admixed with ethyl alcohol or a mixture of ethyl alcohol and water and held under agitation at a temperature of room temperature to a temperature lower than the boiling point of ethyl alcohol or the mixture of water and ethyl alcohol to effect an extraction treatment. The water-ethyl alcohol mixture here contains water and ethyl alcohol preferably in the range of 20:80 to 40:60 by volume ratio and the amount of use thereof is, though not particularly limitative, selected usually in the range of 5 to 20 times by mass relative to the amount of the disintegrated material of the agaricus mycelial bodies. After the extraction treatment, solid matters are removed by centrifugal separation to give an ethyl alcohol extract or a water-ethyl alcohol extract. It is preferable to repeat this extraction procedure several times.

The water and/or ethyl alcohol extracts thus obtained contain a physiologically active substance exhibiting a neurocyte differentiation-inductive activity and acetylcholinesterase activity so that these extracts as such can be used as a neurocyte differentiation-inducing active agent or an acetylcholinesterase active agent. It is optional according to need to use the same as diluted or as concentrated. It is further optional that the extract is spray-dried to recover the physiologically active substance having a neurocyte differentiation-inductive activity and acetylcholinesterase activity in the form of a powder which is used as a neurocyte differentiation-inductive active agent or an acetylcholinesterase active agent.

In this way, the physiologically active substance of the present invention having a neurocyte differentiation-inductive activity and acetylcholinesterase activity can be used as a therapeutic medicine by adequately selecting the medicament form and administration dose in compliance with the intended object.

It is optional that the physiologically active substance of the present invention having a neurocyte differentiation-inductive activity and acetylcholinesterase activity contains other pharmacologically active ingredients in the use thereof. When soybean choline is used in combination with the above-described hot-water extract or ethyl alcohol or water-ethyl alcohol extract, in particular, the neurocyte differentiation-inductive activity and acetylcholinesterase activity contained in the extract can be potentiated. Accordingly, a satisfactory medicament having a neurocyte differentiation-inductive activity and acetylcholinesterase activity can be obtained by compounding soybean choline to the hot-water extract or ethyl alcohol or water-ethyl alcohol extract obtained by the present invention. With regard to the proportion of contents of the soybean choline and the extract there, it is advantageous that the soybean choline is contained in the range of, preferably, 0.05 to 50 parts by mass or, more preferably, 1 to 10 parts by mass relative to 100 parts by mass of the solid matter in the extract.

Besides, the physiologically active substance of the present invention has high safety against living bodies since the active ingredient is an ingredient contained in agaricus which served heretofore as a food. Accordingly, this material can be an additive to a great variety of products including, for example, cosmetic preparations, medicines, foods and others. The concentration of the active ingredient in these products is not particularly limitative and can be appropriately selected within a range to exhibit the desired effect. In a cosmetic preparation, for example, the extract can be contained in about 2 to 20 ppm calculated as solid. When administrated orally, a dose of about 0.2 to 2 mg/kg body weight per day is administrated at one time or in several times.

In the use as a medicine, the medicament forms for use can be, depending on the object of administration, route of administration and others, tablets, capsules, injections, instillations, powders, suppositories, granules, ointments, suspensions, emulsions and others.

Since the physiologically active extract of the present invention exhibits the effect of neurocyte differentiation-inductive activity and effect of acetylcholinesterase activity, it is possible that a food is made a functional food when the same is contained in the food. The food for this purpose can be a kind in which the aforementioned effects of activity are not inhibited without particular limitations. It can be contained in a great variety of foods including, for example, beverages such as fruit juices, refreshing drinks, teas and the like, processed foods such as breads and the like, confectionaries such as candies and the like, precooked foods and others.

The hot-water extract or the ethyl alcohol or water-ethyl alcohol extract from agaricus fungus in the present invention contains a physiologically active substance exhibiting neurocyte differentiation-inductive activity and acetylcholinesterase activity so that a medicament with the aforementioned extract as the effective ingredient can be used as a neurocyte differentiation-inductive activity agent and acetylcholinesterase activity agent having usefulness for therapy of dementia, Alzheimer's disease and others.

The hot-water extract or the ethyl alcohol or water-ethyl alcohol extract from agaricus has high safety and, in addition can be prepared by a simple method so that the range of application thereof is very wide with high utilizability to cosmetic preparations, medicines, foods and others.

In the following, the present invention is described in more detail by way of examples but the present invention is never limited in any way by these examples.

Incidentally, a base culture medium of the following composition was used for the tank culturing in each of the examples.

| | | |
|---|---|---|
| MgSO₄.7H₂O | 0.5 | g |
| FeSO₄.7H₂O | 0.01 | g |
| Yeast extract | 1.0 | g |
| Barley flour | 1.0 | g |
| Sucrose | 30 | g |
| Purified water | 1000 | ml |

### REFERENCE EXAMPLE

Each of the culture mediums A to F prepared by admixing the base culture medium with the additive ingredients of the kind and amount indicated in Table 1 (requisite nitrogen, phosphorus and potassium were provided by organic matters) was inoculated with 5 g of agaricus fungus bodies per liter of the base culture medium (wet basis) and tank culturing was conducted at a temperature of 28-29° C. for 72 hours to prepare agaricus fungus bodies.

TABLE 1

Additives to culture medium

| Sample | Kind | Amount added, g |
|---|---|---|
| A | None | — |
| B | Wheat bran | 1 |
| C | Bagasse | 1 |
| D | Wheat bran/Bagasse | 1/1 |
| E | Wheat bran/Pinewood chips | 1/1 |
| F | Pinewood chips | 1 |

### EXAMPLE 1

The agaricus mycelial samples A to F obtained in Reference Example were separated from the culture medium according to a conventional method and thoroughly washed with water to remove the culture liquid adhering to the surface of the mycelial bodies. Nextly, the mycelial bodies were disintegrated in water by undertaking 5 cycles of ultrasonic treatments with 10 seconds intervals at an output of 100 W, frequency of 50 kHz and temperature of 20° C. In the next place, this disintegrated material was admixed with 10 times by mass of purified water and boiled for 30 minutes. Thereafter, centrifugal separation was conducted for 5 minutes at 3000 rpm and the supernatant thereof was dezymotized by passing a filter of 0.22 μm to prepare samples for testing.

### EXAMPLE 2

The agaricus mycelial samples A to F obtained in Reference Example were separated from the culture medium according to a conventional method and thoroughly washed with water to remove the culture liquid adhering to the surface of the mycelial bodies. Nextly, the mycelial bodies were disintegrated in water by undertaking 5 cycles of ultrasonic treatments with 10 seconds intervals at an output of 100 W, frequency of 50 kHz and temperature of 20° C. In the next place, this disintegrated material was admixed with 10 times by mass of a water-ethyl alcohol mixture containing 70% by volume of ethyl alcohol followed by standing for 7 days at room temperature in a dark room. Thereafter, centrifugal separation was undertaken for 5 minutes at 3000 rpm and the supernatant thereof was dezymotized by passing a filter of 0.22 μm to prepare samples for testing.

### TESTING EXAMPLE

A Dulbecco's modified Eagle culture medium was admixed with 5% by mass of bovine serum and 10% by mass of equine serum followed by the addition of 2×10⁶ cells/ml of PC12 cells as a cell strain originating from the brown cell tumor of rat adrenal medulla, then by the addition of the testing solution obtained in Example 1 or Example 2 in a volume corresponding to 50 μg/ml as solid matter and, in some cases, by the addition of 0.5 μg/ml of soybean choline. Thereafter, culturing was conducted for 2 days at 37° C. in an atmosphere of 5% by volume of carbon dioxide gas.

(1) Measurement of Neurite Extension Rate

Random sampling was made for 300 to 600 cells from the above-described culture, of which the proportion of the cells with neurite extension was recorded in percentages.

(2) Measurement of Acetylcholinesterase Activity

The above-described culture was freed from the culture medium followed by the addition of 50 mmole/liter of a N-2-hydroxyethylpiperazine-N′-2-ethanesulfonic acid buffer solution (pH 7.6), 0.2% by mass of Triton X-100 and 0.12 mole/liter of an aqueous sodium chloride solution and then by the addition of 5.6 mmoles/liter of acetylthiocholine iodide to be agitated for 1 hour at room temperature. Nextly, an acetonitrile solution containing 0.6 mmole/liter of 7-diethylamino-3-(4′-maleimidylphenyl)-4-methylcoumarin was added thereto followed by the addition of 0.2% by mass of Triton X-100, a 1 mmole/liter aqueous EDTA solution and 50 mmoles/liter aqueous sodium acetate solution (pH 5.0) to be agitated for 1 hour at room temperature. The acetylcholinesterase activity was measured thereof by the fluorescence photometric method.

Besides, the neurite extension rate and the acetylcholinesterase activity were measured in the same manner as above for those with addition of, in place of the testing solutions obtained in Example 1 or Example 2, 50 ng/ml of the nerve growth factor (referred to as NGF hereinafter) or 500 ng/ml of soybean choline or for that with addition of nothing. As for the acetylcholinesterase activity, the activity with addition of the NGF was taken as 100% to calculate the relative values for the other treatment groups. The results are shown in Table 2.

TABLE 2

| | Neurite extension | | Acetylcholine- | |
|---|---|---|---|---|
| | rate, % | | sterase activity, % | |
| | Extract | Extract | Extract | Extract |
| | with | with 70% | with | with 70% |
| | hot | ethyl | hot | ethyl |
| Sample added | water | alcohol | water | alcohol |
| A | 23 | 32 | 62 | 67 |
| A + S | 28 | 36 | 75 | 80 |
| B | 88 | 82 | 97 | 86 |
| B + S | 106 | 99 | 114 | 104 |
| C | 86 | 84 | 87 | 91 |
| C + S | 103 | 102 | 105 | 109 |
| D | 82 | 86 | 87 | 86 |
| D + S | 114 | 117 | 122 | 120 |
| E | 64 | 59 | 85 | 76 |
| E + S | 86 | 80 | 119 | 108 |
| F | 68 | 62 | 78 | 68 |
| F + S | 95 | 88 | 112 | 99 |

| | | | |
|---|---|---|---|
| Control | None | 16 | 53 |
| group | NGF | 93 | 100 |
| | Soybean | 26 | 63 |
| | choline | | |

(+ S: Soybean choline used in combination)

It is understood from the results above that all of the hot-water extract and the water-ethyl alcohol extract from agaricus mycelial bodies have excellent neurocyte differentiation-inductive activity. It was noted, in particular, that, in the case of the addition of bagasse or wheat bran to the culture medium, an active substance approximately equivalent to the group with addition of the NGF was contained. Furthermore, it was noted that the activity was increased by 10 to 20%-by the addition of soybean choline over the group with addition of the NGF.

1. A method for the preparation of a physiologically active extract which comprises: disintegrating a mycelial body of agaricus fungus belonging to the basidiomycetous order of matsutake to give a disintegrated material; heating a slurry containing the disintegrated material; and removing solid materials from the slurry.

2. A method for the preparation of a physiologically active extract which comprises: disintegrating a mycelial body of agaricus fungus belonging to the basidiomycetous order of matsutake to give a disintegrated material; keeping a slurry containing the disintegrated material with admixture of ethyl alcohol or a mixture of ethyl alcohol and water; and removing solid materials from the slurry.

3. A method for potentiating the activity of the physiologically active extract obtained by the method of claim 1 which comprises admixing the same with soybean choline.

4. A method for potentiating the activity of the physiologically active extract obtained by the method of claim 2 which comprises admixing the same with soybean choline.
